SEQUENCE LISTING (26 sequences)

SEQ ID NO: 1 - DNA, 18 bp, Artificial Sequence
  Description of Artificial Sequence Synthetic primer
ccggcactcttctggtcc

SEQ ID NO: 2 - DNA, 21 bp, Artificial Sequence
  Description of Artificial Sequence Synthetic primer
tttcctccgcaccatactcgc

SEQ ID NO: 3 - DNA, 22 bp, Artificial Sequence
  Description of Artificial Sequence Synthetic primer
tgggaaatgagaagatccaacg

SEQ ID NO: 4 - DNA, 19 bp, Artificial Sequence
  Description of Artificial Sequence Synthetic primer
cagcacccttcagcctgct

SEQ ID NO: 5 - DNA, 23 bp, Artificial Sequence
  Description of Artificial Sequence Synthetic primer
cacatcccctcacctacattctg

SEQ ID NO: 6 - DNA, 30 bp, Artificial Sequence
  Description of Artificial Sequence Synthetic primer
ttttttcctagaggtcgtggttcactgtga

SEQ ID NO: 7 - DNA, 19 bp, Artificial Sequence
  Description of Artificial Sequence Synthetic primer
ccatcaccccctgacccta

SEQ ID NO: 8 - DNA, 27 bp, Artificial Sequence
  Description of Artificial Sequence Synthetic primer
ttttttttctctgagcaccccgtattg

SEQ ID NO: 9 - DNA, 18 bp, Artificial Sequence
  Description of Artificial Sequence Synthetic primer
agcaccgtgctgacctcc

SEQ ID NO: 10 - DNA, 23 bp, Artificial Sequence
  Description of Artificial Sequence Synthetic primer
ttttccattgttggcacattccg

SEQ ID NO: 11 - DNA, 26 bp, Artificial Sequence
  Description of Artificial Sequence Synthetic primer
ttttttttcgcccctgccttttccta

SEQ ID NO: 12 - DNA, 23 bp, Artificial Sequence
  Description of Artificial Sequence Synthetic primer
cctcgaccctctggaacctatca

SEQ ID NO: 13 - DNA, 19 bp, Artificial Sequence
  Description of Artificial Sequence Synthetic primer
cccctttcccttgtcccac

SEQ ID NO: 14 - DNA, 18 bp, Artificial Sequence
  Description of Artificial Sequence Synthetic primer
catggcgaggagtgcgat

SEQ ID NO: 15 - DNA, 23 bp, Artificial Sequence
  Description of Artificial Sequence Synthetic primer
ctctgggacctcagtttctgcat

SEQ ID NO: 16 - DNA, 20 bp, Artificial Sequence
  Description of Artificial Sequence Synthetic primer
ctgcctagcgaagctgttgt

SEQ ID NO: 17 - DNA, 18 bp, Artificial Sequence
  Description of Artificial Sequence Synthetic primer
ccaggatgaggtgtccgc

SEQ ID NO: 18 - DNA, 19 bp, Artificial Sequence
  Description of Artificial Sequence Synthetic primer
ggctctgcccaggttaagg

SEQ ID NO: 19 - DNA, 26 bp, Artificial Sequence
  Description of Artificial Sequence Synthetic primer
ttttttcagagaagagggtcagtgcg

SEQ ID NO: 20 - DNA, 17 bp, Artificial Sequence
  Description of Artificial Sequence Synthetic primer
agggtcagtggggccga

SEQ ID NO: 21 - DNA, 30 bp, Artificial Sequence
  Description of Artificial Sequence Synthetic primer
ttgaaggaaatgccccattatttagccagg

SEQ ID NO: 22 - DNA, 29 bp, Artificial Sequence
  Description of Artificial Sequence Synthetic primer
tgcttaacaagaccatactacagtgacgt

SEQ ID NO: 23 - DNA, 25 bp, Artificial Sequence
  Description of Artificial Sequence Synthetic primer
aggaggacctgtcctcccagatggt

SEQ ID NO: 24 - DNA, 21 bp, Artificial Sequence
  Description of Artificial Sequence Synthetic primer
ctgtccagccaggagacccca

SEQ ID NO: 25 - DNA, 27 bp, Artificial Sequence
  Description of Artificial Sequence Synthetic primer
cattcttacctggtccccaggacaaag

SEQ ID NO: 26 - DNA, 25 bp, Artificial Sequence
  Description of Artificial Sequence Synthetic primer
gtcctacaaccctgtcacccacatc

(19) United States
(12) Patent Application Publication
(10) Pub. No.: US 2008/0124712 A1
(43) Pub. Date: May 29, 2008

(54) Alpha globin gene dosage assay

(76) Inventors: Feras M. Hantash, Dana Point, CA (US); Weimin Sun, Irvine, CA (US); Shengbiao Wang, Cypress, CA (US)

Correspondence Address: Richard J. Warburg;Foley & Lardner LLP, P.O. Box 80278, San Diego, CA, 92138-0278, US

(21) Appl. No.: 11/588,184

(22) Filed: Oct. 26, 2006

(51) Int. Cl. C12Q 1/68

(52) U.S. Cl. 435/6

(57) ABSTRACT

The methods, compositions and kits disclosed herein relate generally to the detection of nucleic acids and nucleic acid mutations, such as mutations that may be correlated with disease. More specifically, the methods, compositions and kits relate to the detection of mutations in the α-globin gene cluster, to determining the gene dosage of α-globin in a subject, and to diagnosing the subject based on the presence or absence of mutations and/or gene dosage determination.

### FIELD

The invention relates generally to the field of detecting and typing nucleic acids. More specifically, the invention relates to detecting and typing nucleic acid mutations that may be correlated with disease. In particular, the invention relates to the detection of mutations in the α-globin gene cluster, determining α-globin gene dosage, and to diagnosing hemoglobinopathies and related diseases or disorders.

### BACKGROUND

The following discussion of the background of the invention is merely provided to aid the reader in understanding the invention and is not admitted to describe or constitute prior art to the invention.

Hemoglobinopathies, such as various types of thalassemias and sickle cell anemia, are defined as a group of rare, inherited disorders involving abnormal structure of the hemoglobin molecule. In humans, the normal hemoglobin molecule consists of four polypeptide chains, the α-, β-, δ- and γ-globin chains, which are encoded by the α-, β-, δ- and γ-globin genes, respectively. Further, in the human adult, there are three different hemoglobin types made up of different combinations of these globin chains: HbA, HbA2, and HbF. The composition and relative abundance of these hemoglobin types in a normal adult is shown in TABLE 1.

TABLE 1

| Hb Type | Polypeptide Chains | % Total Hb |
|---|---|---|
| HbA | α2β2 | 96-98 |
| HbA₂ | α2δ2 | 2-3 |
| HbF | α2γ2 | <1 |

As shown in TABLE 1, the Ε-globin chain is present in 100% of the hemoglobin types, while the β-globin chain is present in 96-98% of hemoglobin types. Accordingly, disruptions of the α- or β-globin genes, which yield either non-functional or abnormally functional proteins, are the most frequent cause of hemoglobinopathies.

Both the α- and β-globin genes are arranged in gene clusters; the α-globin cluster is located on chromosome 16p13.3 and the β-globin gene cluster is located on chromosome 11p15.5. The α-globin gene cluster contains two highly homologous α-globin genes, HBA1 and HBA2, that are identical at the amino acid level (see e.g., Patrinos G P, et al., Trends Genet 2005, 21:333-38). Accordingly, normal individuals have four copies of α-globin genes, two HBA1 genes and two HBA2 genes, one set on each chromosome.

One category of hemoglobinopathy, the thalassemias, is characterized by a reduced synthesis (and/or function) of one or more of the globin polypeptides. For example, α-thalassemia, a common inherited hemoglobin disorder, is caused by deficient synthesis of α-globin chains (see e.g., Kattamis A C, et al., Am J Hematol 1996, 53:81-91). The clinical phenotype of a carrier of α-thalassemia varies depending on the number of the normal genes. Carriers of three normal α-globin genes are usually asymptomatic, while carriers of two normal α-globin genes have mild anemia. Individuals with only one normal α-globin gene present with hemoglobin H disease characterized by severe anemia, and inheritance of no normal α-globin genes, in most instances, leads to Hb Bart's hydrops fetalis resulting in fetal death.

The severity of β-thalassemia has been found to be modulated by α-globin gene status. For example, co-inheritance of an α-thalassemia allele can ameloriate the phenotype of β-thalassemia while an amplification of α-globin genes can exacerbate the disease.

Over 95% of α-thalassemia alleles are caused by large deletions (termed α-thalassemia deletions) involving either one copy of the HBA genes (e.g.−α3.7 and −α4.2 deletions) or both copies of the HBA genes (e.g.-SEA, -THAI, -FIL or -MED) see e.g., Clark B E, Thein S L, Clin Lab Haematol 2004, 26:159-76; Timmann C. et al., Clin Chem 2005, 51:1711-13). For example, patients with the −α3.7 deletion are missing 3.7 kilobase (“kb”) of DNA from the α-globin gene cluster. The deletion encompasses most of HBA1 and a small portion of HBA2, while the −α4.2 deletion encompasses 4.2 kb and includes all of the HBA2 gene but none of the HBA1 gene. See FIG. 1, where HBA1 is “α−1” and HBA2 is “α−2”. Large deletions causing alpha thalassemia have been detected in the region upstream of the α1 and α2 genes, and include such regulatory regions as HS-40 (see e.g., Viprakasit V. et al., Br J Haematol 2003, 120:867-75). Point mutations and small base pair insertions or deletions have also been detected in the HBA1 and HBA2 genes (see e.g., web site entitled: HbVar: A Database of Human Hemoglobin Variants and Thalassemias). To date, more than 30 different types of α-thalassemia deletions have been recorded.

Traditionally, α-thalassemia deletions have been detected by Southern blot analyses, involving several sets of restriction enzyme digests, radioactive labels and probe detection. Southern blot analysis is both labor intensive and time consuming and as a result, has a high test cost and long test turn-around time.

Other methods, such as the gapped-PCR assay (see e.g., Tan et al., Blood 2001, 98:250-51) have been developed in an attempt to overcome some of the shortcomings of southern blot analysis. Though the gapped-PCR methodology is able to detect seven large deletions in the α-globin gene cluster, neither the Southern nor the gapped-PCR method can detect all reported alpha thalassemia deletions to yield an accurate α-globin gene dosage. See Tan et al. 2001.

### SUMMARY

Provided herein are methods, compositions, and kits directed to determining the number of copies of a gene which are functional, intact or wild-type copies (i.e., gene dosage) in a subject. Such methods are accomplished by amplifying and detecting a segment from each of two or more regions of the gene and determining the dosage of that gene based on a comparison of the amount of the amplification product(s) to one or more control amplification products. Further methods are provided for diagnosing or confirming the presence of disease based on the gene dosage of a particular gene. In preferred embodiments, the gene dosage of α-globin genes are determined and the dosage correlated to the presence of diseases such as, but not limited to, hemoglobinopathies, α- and β-thalassemia, hemoglobin H disease, severe or mild anemia, and Bart's hydrops fetalis.

In a first aspect of the invention, there are provided methods for determining α-globin gene dosage in a subject, wherein the method comprises:
a. preparing a reaction mixture for amplifying nucleic acid comprising:
   (i) genomic nucleic acids from a sample from the subject or a copy of genomic nucleic acids from a sample from the subject,
   (ii) a first and a second set of primers capable of specifically amplifying a first and a second segment of HBA1,
   (iii) a third and a fourth set of primers capable of specifically amplifying a first and a second segment of HBA2,
   (iv) a fifth set of primers capable of specifically amplifying a first segment of SRO,
   (v) a sixth set of primers capable of amplifying a control sequence, wherein the control sequence is outside of the alpha-globin gene cluster;
wherein at least one primer from each set of primers comprises a label;
b. reacting the reaction mixture to generate amplification products;
c. measuring the amount of the various amplification products produced relative to the control amplified product, wherein the α-globin gene dosage is determined.

In another aspect of the invention there are provided methods for diagnosing a disease or confirming the diagnosis of a disease associated with or impacted by α-globin gene dosage level in a subject, wherein the method comprises:
a. determining the number of functional α-globin gene copies present in the genomic nucleic acid of the subject; wherein determining comprises:
b. preparing a reaction mixture for amplifying nucleic acid comprising:
   (i) genomic nucleic acids from a sample from the subject or a copy of genomic nucleic acids from a sample from the subject,
   (ii) a first and a second set of primers capable of specifically amplifying a first and a second segment of HBA1,
   (iii) a third and a fourth set of primers capable of specifically amplifying a first and a second segment of HBA2,
   (iv) a fifth set of primers capable of specifically amplifying a first segment of SRO,
   (v) a sixth set of primers capable of amplifying a control sequence, wherein the control sequence is outside of the alpha-globin gene cluster;
c. amplifying nucleic acids in the amplification mixture to generate amplification products;
d. measuring the amount of the various amplification products produced relative to the control amplified product to determine α-globin gene dosage; and
e. using the gene dosage to diagnose or confirm the diagnosis of a disease associated with or impacted by α-globin gene dosage.

In still another aspect of the invention, there are provided methods for detecting if one or more mutations are present in the alpha-globin gene cluster of a subject, the method comprising:
a. preparing a reaction mixture for amplifying nucleic acid comprising:
   (i) genomic nucleic acids from a sample from the subject or a copy of genomic nucleic acids from a sample from the subject,
   (ii) a first and a second set of primers capable of specifically amplifying a first and a second segment of HBA1,
   (iii) a third and a fourth set of primers capable of specifically amplifying a first and a second segment of HBA2,
   (iv) a fifth set of primers capable of specifically amplifying a first segment of SRO,
   wherein at least one primer from each set comprises a label;
b. amplifying nucleic acids in the amplification mixture to generate amplification products; and
c. detecting the amplification products to determine if one or more mutations are present in the alpha-globin gene cluster of the subject.

In some embodiments of the above aspects of the invention such mutations may include deletions, insertions, rearrangements and point mutations. In particular embodiments, the mutations may be noted relative to the α-globin gene cluster sequence represented by GenBank Accession No. AE006462.

In certain embodiments of the above aspects of the invention, the methods may comprise preparing a reaction mixture for primer directed nucleic acid amplification which includes genomic nucleic acids from a sample of the subject or a copy of genomic nucleic acids from a sample of a subject and amplification primers capable of specifically hybridizing to and amplifying segments of different regions of the α-globin gene cluster. In some embodiments, primers which hybridize to regions of the α-globin gene cluster that are present in wild-type samples but which may be absent in mutants (e.g, α-globin deletion mutants) may be preferred. In other embodiments, the primers may generate wild-type amplicons in wild-type samples, but in mutant samples the amplicons may be: 1) absent; 2) present in different quantity from the wild-type amplicons (e.g., reduced or increased quantity/dosage of amplicons); 3) a different size than the corresponding wild-type amplicon; 4) a different sequence than the corresponding wild-type amplicon; or 5) any combination of 2, 3 and 4.

In some embodiments, primers which target regions such as those deleted in the −α3.7, the −α4.2, -FIL, -SEA, -THAI and/or -MED mutants, in conjunction with primers that target at least one region upstream of the α-globin genes, such as the SRO or an α-globin regulatory region, such as HS-40, are preferred.

In some embodiments, the amplicons to be evaluated may be between about 50 and 1000 bases long, preferably 100 and 500 bases long. In other embodiments, the amplicons may be between about 120 and 340 bases long.

In further embodiments, at least one pair of control oligonucleotide primer pairs (e.g., oligonucleotides capable of specifically hybridizing to and amplifying sequences outside the α-globin gene cluster) may be included. Preferred primer pairs include those capable of hybridizing to and amplifying a segment of a gene selected from the group consisting of: the hexosaminidase A gene, the factor V gene, and the factor II gene.

In some embodiments, the reaction mixture may include one or more of the following, based on GenBank Accession No. AE006462: (A) at least two or at least three oligonucleotides or pairs of oligonucleotides that are capable of specifically hybridizing to and amplifying at least two or at least three different segments of the HBA1 gene of the α-globin cluster, for example, between about nucleotides 165000 to about 167700; (B) at least two or at least three oligonucleotides or pairs of oligonucleotides that are capable of specifically hybridizing to and amplifying at least two or at least three different segments of the HBA2 gene of the α-globin gene cluster, for example between about nucleotides 161850 to about 163800; (C) at least one or at least two oligonucleotides or pairs of oligonucleotides that are capable of amplifying at least one segment common to HBA1 and HBA2; (D) at least one or at least two oligonucleotides or pairs of oligonucleotides that are capable of specifically hybridizing to and amplifying at least one or at least two different segments of the SRO region of the α-globin gene cluster for example, between about nucleotides 92001 to about 112371; (E) at least one oligonucleotide or pair of oligonucleotides that is capable of specifically hybridizing to and amplifying at least one segment of the HS-40 region of the α-globin gene cluster, for example between about nucleotides 103400 to about 103750; (F) at least one oligonucleotide or a pair of oligonucleotides capable of specifically hybridizing to and amplifying at least one segment of the inter-pseudo gene region of the α-globin gene cluster, for example between about nucleotide 156800 to about 157200; and any combination of two or more oligonucleotides or pairs of oligonucleotides.

In certain embodiments, three pairs of oligonucleotides are used to amplify three segments of HBA1, wherein the segments amplified are selected from each of the following three adjacent regions of the gene: the 5′-end region, the middle region, and the 3′-end region. For example, the 5′-end of the HBA1 gene corresponds to about nucleotides 165000 to about 166900 of GenBank Accession No. AE006462; the middle region corresponds to about nucleotides 166901 to 167270; and the 3′-end region corresponds to about nucleotides 167271 to about 167700. Similarly, for HBA2, when three pairs of oligonucleotides are used to amplify three segments of HBA2, one segment is amplified from each of the three adjacent regions (i.e., the 5′-end region, the middle region, and the 3′-end region of HBA2). For HBA2, the 5′-end of the HBA2 gene corresponds to about nucleotides 161850 to about 163100 of GenBank Accession No. AE006462; the middle region corresponds to about nucleotides 163101 to 163470; and the 3′-end region corresponds to about nucleotides 163471 to about 163800.

In other embodiments, two pairs of oligonucleotides are used to amplify two segments of HBA1 or HBA2. In particular embodiments, a first segment is amplified from the 5′-end region and a second segment is amplified, spanning portions of both the middle region and the 3′-end region. In another embodiment, a first segment is amplified from the 3′-end region, and a second segment is amplified, spanning portions of both the 5′-end region and the middle region. In still another embodiment, a first segment is amplified from the 5′-end region, and a second segment is amplified from the 3′-end region. In a further embodiment, a first segment is amplified, spanning portions of both the 5′-end region and the middle region and a second segment is amplified, spanning portions of both the middle region and the 3′-end region.

In particular embodiments, at least one pair of oligonucleotides in (D) i.e., the SRO region and/or (E) i.e., the HS-40 region, above is preferably used in conjunction with one or more pairs of oligonucleotides from any one or more of (F) i.e., inter-pseudo gene region and two or more pairs of oligonucleotides from any two or more of (A) i.e., HBA1 and (B) i.e., HBA2. In other embodiments, oligonucleotides may include one or more of the following SEQ ID NOs: 1-26. In still other embodiments, oligonucleotide pairs may include one or more of the following SEQ ID NO pairs: 1 and 2; 3 and 4; 18 and 20; 9 and 11; 5 and 6; 18 and 19; 9 and 10; 7 and 8; 16 and 17; 12 and 13; 14 and 15.

In certain embodiments, at least one control oligonucleotide or pair of control oligonucleotides (e.g., oligonucleotides capable of specifically hybridizing to sequences outside the α-globin gene cluster) may be included. By way of example, but not by way of limitation such sequences may include one or more of the hexosaminidase A gene sequence, the factor V gene sequence, and the factor II gene sequence. In some embodiments, control oligonucleotides may include one or more of the following: SEQ ID NOs: 21-26. In still other embodiments, oligonucleotide pairs may include one or more of the following SEQ ID NO pairs: 21 and 22; 23 and 24; 25 and 26.

In preferred embodiments, at least one oligonucleotide in each pair may include one or more labels, such as a fluorophore. The label(s) used on oligonucleotides that hybridize to the control sequences may be different than the label(s) used on the oligonucleotides that hybridize to the α-globin gene cluster sequences. In some embodiments, each control label may be different; in other embodiments, different labels may be used for some or all of the α-globin gene cluster oligonucleotides.

In some embodiments, the α-globin gene dosage may be determined and α-globin gene cluster mutations may be detected by evaluating the amplicon size, signal intensity (e.g., fluorescent label intensity), or both size and signal intensity. Signal intensity can be detected by peak height and/or peak area. In particular embodiments, amplicon size and signal intensity may be compared to other α-globin amplicons, to control amplicons, or to both other α-globin amplicons and to control amplicons.

Other embodiments may additionally include nucleic acid sequencing to determine the presence or absence of one or more mutations in the α-globin gene cluster and to determine gene dosage. For example, in some methods, the amplicon size and signal intensity may be evaluated, and then one or more amplicons may be sequenced; for example, amplicons which include segments of the HBA1 and HBA2 genes may be sequenced. These amplicon sequences may then be compared to the HBA1 and HBA2 gene sequences of, for example, the HBA1 and 2 sequences present in GenBank Accession No. AE006462.

The compositions and methods also relate to diagnosing a subject with a disease or disorder based on α-globin gene dosage. For example, the subject may be found to have zero, one, two, three, four, five or more normal, intact, functioning (e.g., wild-type) copies of the α-globin gene, and may be diagnosed, for example, with hemoglobinopathies, Hb Bart's hydrops fetalis, hemoglobin H disease, mild anemia, exacerbated or ameliorated β-thalassemia symptoms or severity. The compositions and methods also relate to confirming the diagnosis of a subject has having α-thalassemia disease resulting from α-globin gene dosage and optionally on other medical criteria.

In another aspect, there are provided oligonucleotides for use as primers in amplifying segments of the α-globin gene cluster or unrelated control gene sequences. Such oligonucleotides may be designed based on the published sequence of the α-globin gene cluster or the control gene sequence. Preferred oligonucleotides are about 10 to about 70 nucleotides, preferably about 10 to about 40 nucleotides, preferably about 15 to about 30 nucleotides. Preferred oligonucleotides comprise any of the sequences set forth in SEQ ID NOs: 1-26 and are about 30, or about 40, or about 50, or about 60, or even up to 70 nucleotides in length. In certain embodiments the oligonucleotide comprise a label, preferably a fluorescent label, a tag sequence, or additional bases that a not complementary to the target sequence may be added to the 5-end of the oligonucleotide to stabilize amplification products or to enhance fragment separation making amplicon discrimination and identification more accurate. It is noted that extensions of sequence at the 3′ end of the primers specified herein preferably are fully complementary to the target sequence, particularly the base at the end 3′, while extensions of sequence at the 5′ of primers specified herein need not be complementary to the target sequence.

In still another aspect, there are provided fragments of α-globin gene cluster generated during the amplification step of particular embodiments of the methods provided herein. Such fragments may be generated by a pair of oligonucleotides selected from the group consisting of: SEQ ID NOs: 1-20. Preferred fragments are those generated by any of the primer pairs selected from the group consisting of: 1 and 2; 3 and 4; 18 and 20; 9 and 11; 5 and 6; 18 and 19; 9 and 10; 7 and 8; 16 and 17; 12 and 13; and 14 and 15.

In yet another aspect, there are provided kits for the determination of α-globin gene dosage and for the detection of mutations in the α-globin gene cluster. Kits may include primers or primer pairs capable of specifically hybridizing to and amplifying segments of the α-globin gene cluster, as well as primer pairs capable of hybridizing to and amplifying control sequences (e.g., sequences other than the as globin gene cluster), such as sequences of one or more of the hexosaminidase A gene, the factor V gene, and the factor II gene. The kits may include one or more of the following primers or primer pairs: SEQ ID NO: 1 and 2; 3 and 4; 18 and 20; 9 and 11; 5 and 6; 18 and 19; 9 and 10; 7 and 8; 16 and 17; 12 and 13; 14 and 15; 21 and 22; 23 and 24; 25 and 26. Kits may also include reagents for amplification, reaction control reagents, and instructions for performing assays and for interpreting results.

### BRIEF DESCRIPTION OF THE DRAWINGS

FIG. 1 shows a schematic of a portion of the α-globin gene cluster (top bar), with portions of exemplary large deletion mutations (gray bars) below. The pseudo genes are represented by hatched boxes; the α-globin genes are represented by the first two black boxes (HBA1 is “α1” HBA2 is “α2”).

FIG. 2 shows a schematic of a portion of the α-globin gene cluster, with the small arrows (below) representing exemplary regions targeted for amplification.

FIG. 3 shows exemplary electropherograms. FIG. 3A shows an α-globin wild-type sample, while FIG. 3B shows a -FIL mutant sample. Control peaks 2, 8 and 14 are underlined.

FIG. 4 shows exemplary electropherograms. The top graph shows an α-globin wild-type sample; the bottom graph shows an anti −α3.7 mutant. Control peaks are 2, 8 and 14.

FIG. 5 shows exemplary electropherograms. The top graph shows an α-globin wild-type sample; the middle graph shows an −α3.7/WT heterozygous mutant; the bottom graph shows an −α3.7/−α3.7 homozygous mutant. Control peaks are 2, 8 and 14.

### DETAILED DESCRIPTION

The compositions, methods and kits described herein relate to determining gene dosage, and to the detection of mutations in genes or sequences that may be correlated with disease. For example, some mutations in the α-globin gene cluster have been correlated with hemoglobinopathies, such as thalassemias, for example α-thalassemia. The methods include, generally, performing multiplex PCR reactions to determine the presence, amount, or both presence and amount of target sequences.

Also disclosed are oligonucleotides, especially primers sequences, which may be useful for the detection of the presence or absence of sequences capable of causing hemoglobinopathies or hemoglobinopathic symptoms.

The present invention is described herein using several definitions, as set forth below and throughout the application.

As used herein, unless otherwise stated, the singular forms “a,” “an,” and “the” includes plural reference. Thus, for example, a reference to “an oligonucleotide” includes a plurality of oligonucleotide molecules, and a reference to “a nucleic acid” is a reference to one or more nucleic acids.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may comprise DNA (e.g., genomic DNA), RNA (e.g., mRNA), cRNA and cDNA, any of which may be amplified to provide amplified nucleic acid. A sample may include nucleic acid in solution or bound to a substrate (e.g., as part of a microarray). A sample may comprise material obtained from an environmental locus (e.g., a body of water, soil, and the like) or material obtained from a fomite (i.e., an inanimate object that serves to transfer pathogens from one host to another).

As used herein, “genomic nucleic acid” refers to some or all of the DNA from the nucleus of a cell. In some embodiments, genomic DNA may include sequence from all or a portion of a single gene or from multiple genes, sequence from one or more chromosomes, or sequence from all chromosomes of a cell. Genomic nucleic acid may be obtained from the nucleus of a cell, or recombinantly produced. Genomic DNA may be transcribed from DNA or RNA isolated directly from a cell nucleus. PCR amplification also may be used.

The term “source of nucleic acid” refers to any sample which contains nucleic acids (RNA or DNA). Particularly preferred sources of target nucleic acids are biological samples including, but not limited to blood, plasma, serum, saliva, cerebral spinal fluid, pleural fluid, milk, lymph, sputum and semen.

As used herein the term “isolated” in reference to a nucleic acid molecule refers to a nucleic acid molecule which is separated from the organisms and biological materials (e.g., blood, cells, serum, plasma, saliva, urine, stool, sputum, nasopharyngeal aspirates and so forth), which are present in the natural source of the nucleic acid molecule. An isolated nucleic acid molecule, such as a cDNA molecule, can be substantially free of other cellular material, or culture medium when produced by recombinant techniques, or substantially free of chemical precursors or other chemicals when chemically synthesized. In some embodiments, nucleic acid molecules encoding polypeptide/proteins may also be isolated or purified. Methods of nucleic acid isolation are well known in the art and may include total nucleic acid isolation/purification methods, RNA-specific isolation/purification methods or DNA-specific isolation/purification methods. In certain embodiments, particularly in the case where one is evaluating a nucleic acid sample from an individual, the nucleic acid from the individual may be used in cude form (i.e. with little to no purification).

As used herein, the term “subject” is refers to an animal, preferably a mammal, more preferably a human. The term “subject” and “patient” may be used interchangeably.

As used herein, the term “α-globin gene cluster” or “alpha-globin gene cluster” referd to the region on human chromosome 16p13.3 which includes, but is not limited to, upstream sequences such as the SRO, regulatory sequences such as the HS-40 region, pseudo genes, the embryonic α-globin gene (ζ2-globin), two fetal/adult genes (α1 and α2 or HBA1 and HBA2, respectively), and the intervening sequences (e.g., sequences between and among the above-named regions or genes). An exemplary, representative α-globin gene cluster is shown by GenBank Accession No: AE006462.

HBA1 and HBA2 refer to the two highly homologous “α-globin genes” (or “alpha-globins genes” also known as “α1” and “α2”), which when wild-type, are identical at the amino acid level. As used herein, HBA1 and HBA2 each refers to upstream sequence, exons, introns, and downstream sequence. For example, an within GenBank Accession No: AE006462 between about nucleotides 165000 to about 167700. An exemplary HBA2 gene sequence, including some upstream sequence, is encompassed within GenBank Accession No: AE006462 between about nucleotides 161850 to about 163800.

As used herein “α-globin pseudo genes” or “pseudo genes” refer to the sequences including the ψα1, ψα2 and the ζ1 pseudo genes. The “inter-pseudo gene region” as used herein refers to the region between the ψα1 and ψα2 pseudo genes. An exemplary, representative inter-pseudo gene region is encompassed within GenBank Accession No. AE006462 between about nucleotides 156800 and 157200.

As used herein “SRO” refers to the “Shortest Region of Overlap” of large deletions in the α-globin gene cluster. This region is described in a report by Vipraskasit et al. (Br J Haematology 120:867-75, 2003) which compared known α-thalassemia deletion mutations involving sequences upstream of the alpha globin genes. The SRO was defined in this report by aligning the deleted sequences and identifying the region of deleted sequence common to all. This region is approximately 20 kb in length, spanning a region located approximately 31-51 kb upstream of the embryonic α-globin gene (the ζ-globin gene). This region includes a number of DNAse I hypersensitive sites, such as HS-46, HS-40, HS-38, HS-36, and HS-33. An exemplary, representative SRO is encompassed within GenBank Accession No: AE006462 between about nucleotides 92001 and 112371.

As used herein “HS-40” or “HS-40 region” (also known as a “Hypersensitive Site” or “alpha MRE”) refers to the approximately 350-nucleotide, highly conserved, upstream, major regulatory element of the α-globin gene cluster. This region is located approximately 40 kb upstream of the embryonic α-globin gene (the ζ-globin gene). An exemplary, representative HS-40 region is encompassed within GenBank Accession No: AE006462 between about nucleotides 103400 and 103750.

As used herein “control target” or “control region” or “control sequence” or “standard” refers to sequences that are distinct from to the α-globin gene cluster, and that may be used as a standard for gene dosage determination. In some embodiments, the standard or control target is present in two copies per genome. By way of example, but not by way of limitation, control sequences may include regions of the hexosaminidase A gene (see e.g., GenBank Accession No. AC009690), regions of the Factor V gene (see e.g., GenBank Accession No. Z99572), or regions of the Factor II gene (see e.g., GenBank Accession No. M17262). In some embodiments, the hexosaminidase A gene region between about nucleotides 134400 to about 134600, such as from about 134420 to about 134559 of GenBank Accession No. AC009690 may be targeted as a control sequence; in other embodiments the Factor V gene region between about nucleotides 62750 to about 63100, such as from about 62821 to about 63032 of GenBank Accession No. Z99572 may be targeted as a control sequence; in still other embodiments, the Factor II gene region between about nucleotides 6350 to about 6800, such as from about 6401 to about 6732 of GenBank Accession No. M17262 may be targeted as a control sequence.

A “mutation” is meant to encompass at least a single nucleotide variation in a nucleic acid sequence relative to the normal sequence or wild-type sequence. A mutation may include a substitution, a deletion, a duplication, an inversion or an insertion; such mutations may encompass a single nucleotide or multiple nucleotides. For example, a deletion may involve a single base or kilobases; likewise, an insertion or duplication may involve a single base or kilobases, etc.

With respect to an encoded polypeptide, a mutation may be “silent” and result in no change in the encoded polypeptide sequence or a mutation may result in a change in the encoded polypeptide sequence. For example, a mutation may result in a substitution in the encoded polypeptide sequence. A mutation may result in a frameshift with respect to the encoded polypeptide sequence. A mutation, such as a deletion mutation, may result in the elimination of an entire polypeptide or a region of a polypeptide, or a mutation such as a duplication may result in the addition of an entire polypeptide or a region of a polypeptide.

By way of example, but not by way of limitation, mutations in the human as globin gene cluster may include large deletions involving one or both of the HBA1 or HBA2 genes (e.g., −α03.7, −α4.2, -SEA, -THAI, -FIL, or -MED), or in a region upstream of the α-globin genes, such as the SRO, or an α-globin regulatory region, such as HS-40, or combinations of the these regions. Mutations may be determined relative to the α-globin gene cluster sequence of GenBank Accession No. AE006462.

As used herein, the term “α-globin gene dosage” refers to the number of wild-type, intact, or functional copies of the α-globin gene that are present in a subject or a sample. For example, a normal human subject has four copies of α-globin; two copies (i.e., HBA1 and HBA2) on each chromosome. Thus, a normal human subject would have an alpha-globin gene dosage of four. Some subjects may have a gene dosage of zero, one, two, three, four, five or more. In some embodiments, gene dosage may be correlated with disease, disease states, syndromes, symptoms or conditions such as, but not limited to, hemoglobinopathies, Bart's hydrops fetalis, hemoglobin H disease, severe anemia, mild anemia, alpha thalassemia, ameliorated phenotypes or severity of β-thalassemia, and exacerbated phenotypes or severity of β-thalassemia.

An oligonucleotide is a nucleic acid that includes at least two nucleotides. Oligonucleotides used in the methods disclosed herein typically include at least about ten (10) nucleotides and more typically at least about fifteen (15) nucleotides. In some embodiments, preferred oligonucleotides may be from about 15, to about 20, to about 30, or to about 40 nucleotides, or to about 50 nucleotides, or to about 60 nucleotides, or even to about 70 nucleotides. An oligonucleotide may be designed to function as a “primer.”

A “primer” is a short nucleic acid, usually a single-stranded DNA oligonucleotide, which may be annealed to a target polynucleotide by complementary base-pairing. The primer may then be extended along the target DNA or RNA strand by a polymerase enzyme, such as a DNA polymerase enzyme. Exemplary primers include, but are not limited to, SEQ ID NOs: 1-26, complements, and variants, which may include the addition or subtraction of from about 1 to about 7 nucleotides from either the 5′ end the 3′ end or both, and substitutions, insertions or deletions of up to about 3 nucleotides. It is noted that extensions of sequence at the 3′ end of the primers specified herein preferably are fully complementary to the target sequence, particularly at the end 3′ base, while extensions of sequence at the 5′ of primers specified herein need not be complementary to the target sequence.

The phrases “a set of primers” and “a primer pair” are used interchangeably herein and generally refer to two oligonucleotides that are used together in the amplification of a segment of nucleic acid. For example, primer pairs or primer sets can be used for amplification (and identification) of a nucleic acid sequence (e.g., by the polymerase chain reaction (“PCR”)). A first primer set may include two primers (e.g., a forward and a reverse primer). A second primer set may include two completely different primers than the first primer set, or may include a primer or primers in common with the first primer set. For example, primer sets A and B may share a common forward primer. Primer set A (the common forward primer and reverse primer A) may yield an amplification product of 100 nucleotides. Primer set B (the common forward primer and a reverse primer B) may yield an amplification product of 150 nucleotides. Thus, these two primer sets amplify different segments; the A segment of 100 bases, and the B segment, which encompasses the A segment plus an additional 50 nucleotides. By way of example, but not by way of limitation, primers pairs may include SEQ ID NO. pairs: 1 and 2; 3 and 4; 18 and 20; 9 and 11; 5 and 6; 18 and 19; 9 and 10; 7 and 8; 16 and 17; 12 and 13; 14 and 15; 21 and 22; 23 and 24; 25 and 26.

An oligonucleotide may be designed to function as a “probe.” A “probe” refers to an oligonucleotide, its complements, or fragments thereof, which is used to detect identical, allelic or related nucleic acid sequences.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of nucleotide matches between at least two polynucleotide sequences aligned using a standardized algorithm (e.g., BLAST) using default gap penalty settings.

An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Preferably, specific hybridization results in complexes that remain hybridized under moderately or even more preferably stringent washing conditions. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm, for example nearest-neighbor parameters, and conditions for nucleic acid hybridization are known in the art.

As used herein “target,” or “target nucleic acid” refers to a nucleic acid molecule containing a sequence that has at least partial complementarity with an oligonucleotide, for example a probe or a primer. A “target” sequence may include a part of a gene or genome. A “target” may be wild-type or mutant.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These terms also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

The amplification methods described herein may include end-point monitoring. End-point monitoring refers to the detection and/or evaluation of amplification products when the reaction is stopped, either temporarily or permanently. The amplification methods described herein may include “real-time monitoring” or “continuous monitoring.” These terms refer to monitoring multiple times during PCR amplification, preferably during temperature transitions, and more preferably obtaining at least one data point for each temperature transition. The term “homogeneous detection assay” is used to describe an assay that includes coupled amplification and detection, which may include “real-time monitoring” or “continuous monitoring.”

The amplification methods described herein may include “multiplex amplification.” As used herein a multiplex amplification reaction (e.g., a multiplex PCR reaction) refers to a PCR reaction where more than one primer set is included in the reaction mixture, allowing two or more different targets to be amplified by the PCR in a single reaction vessel (e.g., in a tube or in a well of a microtiter plate). By way of example but not by way of limitation, a multiplex reaction may include a primer set capable of amplifying a segment of a gene such as HBA1, a primer set capable of amplifying a segment of a gene such as HBA2, a primer set capable of amplifying a segment of the α-globin gene cluster SRO region, a primer set capable of amplifying a segment of the HS-40 region, a primer set capable of amplifying a segment between the α-globin gene cluster pseudo genes, and a primer set capable of amplifying a control sequence or combination thereof.

Amplification of nucleic acids may include amplification of nucleic acids or segments or subregions of these nucleic acids. For example, amplification may include amplifying segments of nucleic acids between about 30 and 50, between about 50 and 100, or between about 100 and 500 nucleotides in length or between about 500 to 1000 nucleotides in length; for example, in some embodiments, amplification products may be between about 120 to about 340 nucleotides in length. The length of the amplicon may be pre-determined by selecting the proper primer sequences.

In some embodiments, the oligonucleotides are labeled. Probes or primers may include oligonucleotides which have been attached to a detectable label or reporter molecule. Typical labels include fluorescent dyes, quenchers, radioactive isotopes, ligands, scintillation agents, chemiluminescent agents, and enzymes. For example, oligonucleotide which function as primers may be labeled with a reporter that emits a detectable signal (e.g., a fluorophore). Accordingly, amplification products which include the labeled primers will also be labeled and may be detected by any of a variety of methods known in the art. Further, the intensity of the labeled products may be compared, for example in a multiplex reaction, to the intensity of each of the other amplicons, or to the intensity of a control amplicon or amplicons.

In some methods, label or signal intensity may be correlated to the amount of amplicon present in the reaction, and/or to the amount of target in the starting sample. For example, a multiplex reaction may result in five different “test” amplicons and a control amplicon. The intensity of the control amplicon is 100 units and represents, for example, two copies of a gene. If the intensity of amplicons from test samples 1-5 is 0, 50, 100, 150 and 200 units, respectively, sample 1 would contain no amplicon (and may indicate no copies of the target gene); sample 2 would contain half the amount of amplicon as the control (and may indicate half the number of target gene copies (e.g., a single copy of a gene)); sample 3 would contain the same amount of amplicon as the control (and may indicate two copies of the target gene); sample 4 would contain 1.5 times the amount of amplicon as the control (and may indicate 3 copies of the target gene); and sample 5 would contain 2 times the amount of amplicon as the control (and may indicate 4 copies of the target gene).

In some embodiments, each different fluorescent dye may emit a signal that can be distinguished from a signal emitted by any other of the different fluorescent dyes that are used to label the oligonucleotides. For example, the different fluorescent dyes may have wavelength emission maximums all of which differ from each other by at least about 5 nm (preferably by least about 10 nm). In some embodiments, each different fluorescent dye is excited by different wavelength energies. For example, the different fluorescent dyes may have wavelength absorption maximums all of which differ from each other by at least about 5 nm (preferably by at least about 10 nm).

As used herein, “labels” or “reporter molecules” are chemical or biochemical moieties useful for labeling a nucleic acid, amino acid, or antibody. “Labels” and “reporter molecules” include fluorescent agents, chemiluminescent agents, chromogenic agents, quenching agents, radionuclides, enzymes, substrates, cofactors, scintillation agents, inhibitors, magnetic particles, and other moieties known in the art. “Labels” or “reporter molecules” are capable of generating a measurable signal and may be covalently or noncovalently joined to an oligonucleotide.

As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: isomer-free succinimidyl ester of 6-carboxy-2′,4,4′,5′,7,7′-hexachlorofluorescein (HEX): 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 6-FAM (6-Carboxyfluorescein); 56-FAM; 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; β-Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3nm; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP(S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamine-IsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof. In some embodiments, HEX and FAM may be preferred.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

In other embodiments, the oligonucleotides of the disclosed methods may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

The oligonucleotide of the present methods may be labeled with a donor fluorophore and an acceptor fluorophore (or quencher dye) that are present in the oligonucleotides at positions that are suitable to permit FRET (or quenching). Labeled oligonucleotides that are suitable for the present methods may include but are not limited to oligonucleotides designed to function as LightCycler primers or probes, Taqman® Probes, Molecular Beacon Probes, Amplifluor® Primers, Scorpion® Primers, and LuX™ Primers.

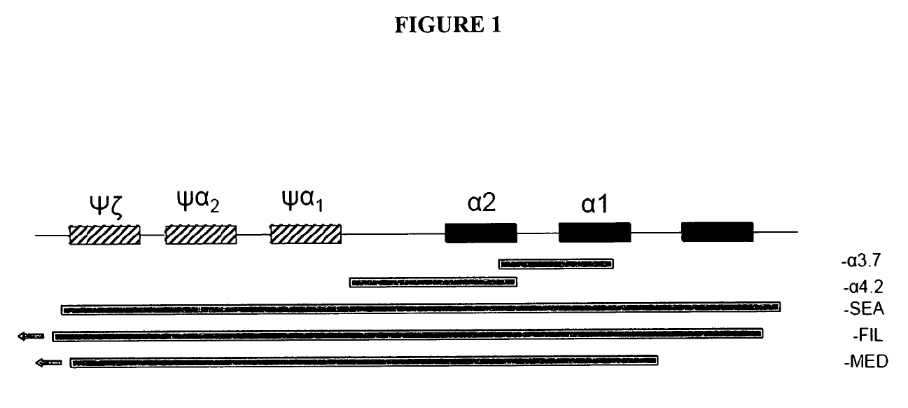

As discussed, mutations in the α-globin gene cluster that result in a lack of α-globin gene function may include deletions, insertions or point mutations. For example, a number of large deletions have been identified in alpha thalassemia patients. These deletions may span mainly one of the α-globin genes (e.g.−α4.2 deletion), span parts of both HBA1 and HBA2 (e.g.−α3.7) or may span both copies of the α-globin genes (e.g. -THAI, -FIL or -MED) (see e.g., FIG. 1). Deletions have also been identified in the HS-40 regulatory region. Point mutations, small base pair insertions and deletions that affect α-globin gene function have also been detected in HBA1 and HBA2 genes. Both the large deletions as well as the smaller point mutation and insertion/deletion mutations have been shown to cause the loss of function of an α-globin gene. Accordingly, detection of both types of mutations would provide a more reliable assessment of the number of functional α-globin genes in a subject.

The examples below illustrate methods to detect both large deletions and small base pair insertions and deletions, if present in a patient sample. Using a single-tube, multiplex, fluorescent PCR assay which combines up to twenty-six different primers, mutations in the α-globin gene cluster can be detected, and the dosage of the α-globin gene in a patient can be more accurately determined in a single amplification. Thus, in a preferred embodiment, the multiplex PCR assay includes 26 different primers which generate amplification products spanning the α-globin gene cluster, including the SRO and HS-40 regions, and also amplify control sequences. In order to distinguish the different amplicons, amplification primers are designed such that the resulting amplicons are different sizes. Additionally, at least two different labels may be used in the different amplification primer sets (e.g., one label for the control sequences and a different label for the α-globin related sequences). Thus, when the amplified fragments are separated, for example, on an automated DNA analyzer, both the size and the color of the fragment will allow a user to distinguish and identify each amplicon. In addition, by noting differences in label intensity as compared to controls, the presence of large deletions and duplications/insertions can be detected. For example, an increase in signal intensity by about 50% would indicate an insertion/duplication of the target region, while a decrease in signal intensity by about 50% would indicate a deletion of that region. Additionally, the optional step of sequencing any or all amplicons, or the HBA1 and 2 genes specifically, will allow for the precise identification or verification of any transition, transversion, small insertion or deletion mutations.

The diversity and utility of the compositions, methods and kits are demonstrated in the following examples which are meant to be instructive and not limiting. Although the following example demonstrates the features of the methods and compositions using the α-globin gene cluster, it is understood that the methods could be used to detect mutations in other genes or regions of the genome, in human or other organisms.

### EXAMPLE 1

### Amplification Assay for Detection of Mutations in the α-Globin Gene Cluster

1. Amplification Primers

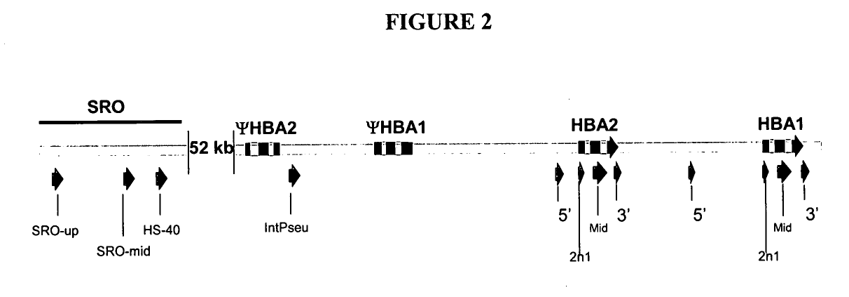

The regions of the α-globin gene cluster that are targeted for amplification are show graphically in FIG. 2, and include: 1) three primer sets to amplify a segment from each of three different regions of the HBA1 gene; 2) three primer sets to amplify three different regions of the HBA2 gene; 3) a primer set to amplify a region common to both the HBA1 and HBA2 genes; 4) two primer sets to amplify different regions of the SRO; 5) one primer set to amplify a region of the HS-40; and 6) a primer set to amplify a region between the two α-globin pseudo genes.

As used herein, a segment “common” to the HBA1 gene and the HBA2 gene includes any sequence that is present in both of these genes. A preferred common segment to the HBA1 and HBA2 genes is the “2n1” segment. FIG. 2 is a schematic depicting the α-globin gene cluster and the segments selected for amplification (small arrows). TABLE 2 provides details regarding the amplification primers. Column 1 of TABLE 2 shows the SEQ ID NO for each primer. Column 2 gives a primer name which designates the target to be amplified. Column 3 shows the sequence, including labels if any. Column 4 provides the location of the primer sequence within the listed reference GenBank sequence. Underlined, italicized bases indicate extra bases that were added to enhance fragment separation making amplicon discrimination and identification more accurate. TABLE 3 shows which primers are used to create specific amplicons.

Primers were synthesized at 1.0 μM scale and HPLC purified (IDT). Primers were then quantitated and stored as 100 μM stock solutions at −60° to −80° C.

TABLE 2

| | | | |
|---|---|---|---|
| | | | GenBank Ac- |
| SEQ | | | cession No. |
| ID | Sequence | | and Primer |
| NO: | Name | Sequence | Location |

| | | | |
|---|---|---|---|
| | | | No. |
| | | | AE006462 |
| 1 | HBA2n1F | 5′-/56- | 162870- |
| | | FAM\CCGGCACTCTTCTGGTCC-3′ | 162887; |
| | | | 166674- |
| | | | 166691 |
| 2 | HBA2n1R | 5′-TTTCCTCCGCACCATACTCGC- | 162996- |
| | | 3′ | 162979; |
| | | | 166800- |
| | | | 166783 |
| 3 | HBA1UpF2 | 5′-\56- | 165119- |
| | | FAM\TGGGAAATGAGAAGATCCA | 165139 |
| | | ACG-3′ | |
| 4 | HBA1UpR2 | 5′-CAGCACCCTTCAGCCTGCT-3′ | 165278- |
| | | | 165260 |
| 5 | HBA2UpV4F | 5′-\56- | 161916- |
| | | FAM\CACATCCCCTCACCTACATT | 161938 |
| | | CTG-3′ | |
| 6 | HBA2UpV4R | 5′- | 162084- |
| | | TTTTTTCCTAGAGGTCGTGGTTCA | 162061 |
| | | CTGTGA-3′ | |
| 7 | SROupF | 5′-\56- | 92604- |
| | | FAM\CCATCACCCCCTGACCCTA- | 92622 |
| | | 3′ | |
| 8 | SROupR | 5′- | 92779- |
| | | TTTTTTTTCTCTGAGCACCCCGTA | 92761 |
| | | TTG-3′ | |
| 9 | AG3F1 | 5′-\56- | 163570- |
| | | FAM\AGCACCGTGCTGACCTCC- | 163587; |
| | | 3′ | 167381- |
| | | | 167398 |
| 10 | HBA2DnR | 5′- | 163757- |
| | | TTTTCCATTGTTGGCACATTCCG- | 163739 |
| | | 3′ | |
| 11 | HBA1DnR | 5′- | 167642- |
| | | TTTTTTTTCGCCCCTGCCTTTTCCT | 167625 |
| | | A-3′ | |
| 12 | HS40F | 5′-\56- | 103491- |
| | | FAM\CCTCGACCCTCTGGAACCTA | 103513 |
| | | TCA-3′ | |
| 13 | HS40R | 5′-CCCCTTTCCCTTGTCCCAC-3′ | 103695- |
| | | | 103677 |
| 14 | IntPseuF | 5′-\56- | 156896- |
| | | FAM\CATGGCGAGGAGTGCGAT-3′ | 156913 |
| 15 | IntPseuR | 5′- | 157126- |
| | | CTCTGGGACCTCAGTTTCTGCAT- | 157104 |
| | | 3′ | |
| 16 | SROmidF | 5′-\56- | 102401- |
| | | FAM\CTGCCTAGCGAAGCTGTTGT- | 102420 |
| | | 3′ | |
| 17 | SROmidR | 5′-CCAGGATGAGGTGTCCGC-3′ | 102632- |
| | | | 102649 |
| 18 | AGmidF3 | 5′-GGCTCTGCCCAGGTTAAGG-3′ | 163182- |
| | | | 163200; |
| | | | 166986- |
| | | | 167004 |
| 19 | HBA2midR | 5′-\56- | 163465- |
| | | FAM\TTTTTTCAGAGAAGAGGGTC | 163446 |
| | | AGTGCG-3′ | |
| 20 | HBA1midR | 5′-\56- | 167268- |
| | | FAM\AGGGTCAGTGGGGCCGA-3′ | 167252 |
| | | | No. Z99572 |
| 21 | F5F | 5′ - HEX-TTG AAG GAA ATG | 62821- |
| | | CCC CAT TAT TTA GCC AGG - | 62850 |
| | | 3′ | |
| 22 | F5R | 5′ - TGC TTA ACA AGA CCA | 63004- |
| | | TAC TAC AGT GAC GT - 3′ | 63032 |
| | | | No. M17262 |
| 23 | F2F | 5′ - 6-FAM-AGG AGG ACC | 6401- |
| | | TGT CCT CCC AGA TGG T - | 6425 |
| | | 3′ | |
| 24 | F2R | 5′ - CTG TCC AGC CAG GAG | 6712- |
| | | ACC CCA - 3′ | 6732 |
| | | | No. |
| | | | AC009690 |
| 25 | TSF | 5′ - HEX-CAT TCT TAC CTG | 134420- |
| | | GTC CCC AGG ACA AAG - 3′ | 134446 |
| 26 | TSR | 5′ - GTC CTA CAA CCC TGT | 134535- |
| | | CAC CCA CAT C - 3′ | 134559 |

TABLE 3

| SEQ ID | | | |
|---|---|---|---|
| NOs | AMPLICON | SIZE | LABEL |
| 1 + 2 | HBA1/HBA2 | 130 | FAM |
| | common | | |
| 3 + 4 | HBA1 5′ segment | 161 | FAM |
| 18 + 20 | HBA1 mid segment | 283 | FAM |
| 9 + 11 | HBA1 3′ segment | 270 | FAM |
| 5 + 6 | HBA2 5′ segment | 175 | FAM |
| 18 + 19 | HBA2 mid segment | 290 | FAM |
| 9 + 10 | HBA2 3′ segment | 192 | FAM |
| 7 + 8 | SRO -up | 184 | FAM |
| 16 + 17 | SRO -mid | 249 | FAM |
| 12 + 13 | HS-40 | 205 | FAM |
| 14 + 15 | Inter-pseudo gene | 231 | FAM |
| 21 + 22 | Control: F5 | 212 | HEX |
| 23 + 24 | Control: F2 | 332 | FAM |
| 25 + 26 | Control: TS | 140 | HEX |

2. Reaction Mixes

For ease of use, reaction mixes were made. This is optional, as each individual component may be added to the reaction vessel separately.

a. 25 mM dNTPs

100 mM stock solutions of dATP, dCTP, dGTP, and dTTP (e.g., Pharmacia, # 27-2035-01) were thawed, and 50 μL of each dNTP was added to a sterile microfuge tube. The tube was vortexed for 2 seconds to mix. The tubes were then microcentrifuged at maximum speed for 2 seconds. Stock solutions were stored at −10° C. or colder.

b. Primer Mix

TABLE 4 shows an exemplary mixture of primers for the amplification of the α-globin gene cluster targets and controls in a single multiplex reaction. The stock primer mixture may be aliquoted and stored at −20° C.

TABLE 4

| | | | |
|---|---|---|---|
| Primer Name | | μL/2000 | Final μM |
| (SEQ ID NO) | μL/Rxn | Rxns | Concentration |

| | | | |
|---|---|---|---|
| HBA2n1F (1) | 0.02 | 40 | 0.08 |
| HBA2n1R (2) | 0.02 | 40 | 0.08 |
| TSF (25) | 0.1 | 200 | 0.4 |
| TSR (26) | 0.1 | 200 | 0.4 |
| HBA1UpF2 (3) | 0.04 | 80 | 0.16 |
| HBA1UpR2 (4) | 0.04 | 80 | 0.16 |
| HBA2DnR (10) | 0.05 | 100 | 0.2 |
| HBA2V4F (5) | 0.04 | 80 | 0.16 |
| HBA2V4R (6) | 0.04 | 80 | 0.16 |
| AG3F1 (9) | 0.05 | 100 | 0.2 |
| HBA1DnR (11) | 0.05 | 100 | 0.2 |
| SROupF (7) | 0.025 | 50 | 0.1 |
| SROupR (8) | 0.025 | 50 | 0.1 |
| HS40F (12) | 0.025 | 50 | 0.1 |
| HS40R (13) | 0.025 | 50 | 0.1 |
| F5F (21) | 0.05 | 100 | 0.2 |
| F5R (22) | 0.05 | 100 | 0.2 |
| SROmidF (16) | 0.05 | 100 | 0.2 |
| SROmidR (17) | 0.05 | 100 | 0.2 |
| F2F (23) | 0.07 | 140 | 0.28 |
| F2R (24) | 0.07 | 140 | 0.28 |
| AGmidF32 (18) | 0.08 | 160 | 0.32 |
| HBA1midR (20) | 0.08 | 160 | 0.32 |
| HBA2midR (19) | 0.08 | 160 | 0.32 |
| IntpseuF (14) | 0.025 | 50 | 0.1 |
| IntpseuR (15) | 0.025 | 50 | 0.1 |
| TOTAL | 1.28 | 2560 | |

c. PCR Mixes

PCR mixes may also be made in advance and frozen as stocks. The following PCR pre-mix was made (TABLE 5).

TABLE 5

| Reagent | μL/Rxn | μL/2000 | Final Conc | UNIT |
|---|---|---|---|---|
| 10X Roche w/o MgCl₂ | 3.75 | 7500 | 1.5 | X |
| MgCl₂ | 2 | 4000 | 2 | mM |
| 5X GC-rich Buffer | 5 | 10000 | 1 | X |
| dNTPs (25 mM) | 0.2 | 400 | 0.2 | mM |
| dH₂O | 10.57 | 21140 | | |
| Primer Mix | 1.28 | 2560 | | |
| TOTAL | 22.8 | 45600 | | |

3. Target DNA Preparation

Sample or target DNA may prepared from whole blood, blood fractions, amniotic fluid, cultured cells, chorionic villi, by any number of methods known in the art. For example, phenol:chloroform nucleic acid purification methods may be used, or commercially available kits and reagents, such as those produced and sold by Gentra Systems (Minneapolis, Minn.), Promega Corporation (Madison, Wis.), or Qiagen, (e.g., Qiagen BioRobot 9640 Genomic DNA preparation reagents, Qiagen, Valencia, Calif.) may be used. In general, between about 10 ng and 100 μg DNA may be used for each reaction.

4. Reaction

Using the mixes described above, TABLE 6 provides PCR reaction conditions. The table below shows the setup for 40 samples.

TABLE 6

| Reagents | 1 Rxn (μL) | 40 rxns |
|---|---|---|
| AG PCR MIX | 22.8 | 912 |
| ENZYME | 0.2 | 8 |
| TOTAL | 23 | 920 |

Twenty-three μL of the PCR/enzyme mix may be dispensed into each reaction vessel (e.g, a well of a microtiter plate). Two μL of DNA sample (10 ng and 100 μg) are added to each well. Samples may be run in duplicate, and controls (e.g., no DNA; samples of known genotype, etc.) may also be run.

5. PCR Cycling Parameters

Cycling parameters may differ depending on the thermocycler used. For example, TABLE 7 shows exemplary cycling parameters for an MJ Research brand thermocycler, while TABLE 8 shows exemplary cycling parameters for the ABI 7900 thermocycler.

TABLE 7
PCR Cycling Parameters
(MJ Research PTC 200 or 225)

| | | |
|---|---|---|
| 1 | 95° C. | 10 min. |
| 2 | 94° C. | 30 sec |
| 3 | 58° C. | 30 sec |
| 4 | 72° C. | 1 min |
| 5 | go to step 2 | 23 cycles* |
| 6 | 72° C. | 5 min |
| 7 | 60° C. | 3 hours |
| 8 | 25° C. | Hold |

*Typically 23 cycles are used. However, 21-23 cycles can be used.

TABLE 8
PCR Cycling Parameters (ABI 9700)*

| | | |
|---|---|---|
| 1 | 95° C. | 10 min. |
| 2 | 94° C. | 30 sec |
| 3 | 56° C. | 45 sec |
| 4 | 72° C. | 1 min |
| 5 | go to step 2 | 21 cycles** |
| 6 | 72° C. | 5 min |
| 7 | 60° C. | 70 min |
| 8 | 60° C. | 70 min |
| 9 | 60° C. | 70 min |
| 10 | 25° C. | Hold |

*Note about ABI 9700: due to programming limitations of the instrument, incubation at 60° is performed in three consecutive steps.
**Typically 23 cycles are used. However, 21-23 cycles can be used.

6. Results

Amplicons were denatured (e.g., by HiDi Formamide) and were analyzed on an ABI 3100 automated DNA analyzer (Applied Biosystems, Foster City, Calif.) to determine both size and color. The expected and observed amplicon size, as well as dye color, are presented TABLE 9, below. Note that assays may be performed using size marker controls such as GeneScan-350 ROX Size Standard (Applied Biosystems, Foster City, Calif.).

TABLE 9

| Amplicon | | | |
|---|---|---|---|
| (Peak number) | Size | Observed* | DYE |
| HBA1/HBA2 common | 130 | 126.5 | Blue |
| (1) | | | |
| Control: TS | 140 | 137.5 | Green |
| (2) | | | |
| HBA1 5′ segment | 161 | 159.9 | Blue |
| (3) | | | |
| HBA2 5′ segment | 175 | 172.9 | Blue |
| (4) | | | |
| SRO-up | 184 | 181.8 | Blue |
| (5) | | | |
| HBA2 3′ segment | 192 | 188.3 | Blue |
| (6) | | | |
| HS-40 | 205 | 205 | Blue |
| (7) | | | |
| Control: F5 | 212 | 209.6 | Green |
| (8) | | | |
| Inter-pseudo gene | 231 | 230.5 | Blue |
| (9) | | | |
| SRO-mid | 249 | 245.1 | Blue |
| (10) | | | |
| HBA1 3′ segment | 270 | 266.1 | Blue |
| (11) | | | |
| HBA1 mid segment | 283 | 276.3 | Blue |
| (12) | | | |
| HBA2 mid segment | 290 | 284.0 | Blue |
| (13) | | | |
| Control: F2 | 332 | 331.9 | Blue |
| (14) | | | |

*Size of the fragments is affected by mobility shift caused by the incorporated fluorescent-labeled dye.
Blue = FAM
Green = HEX

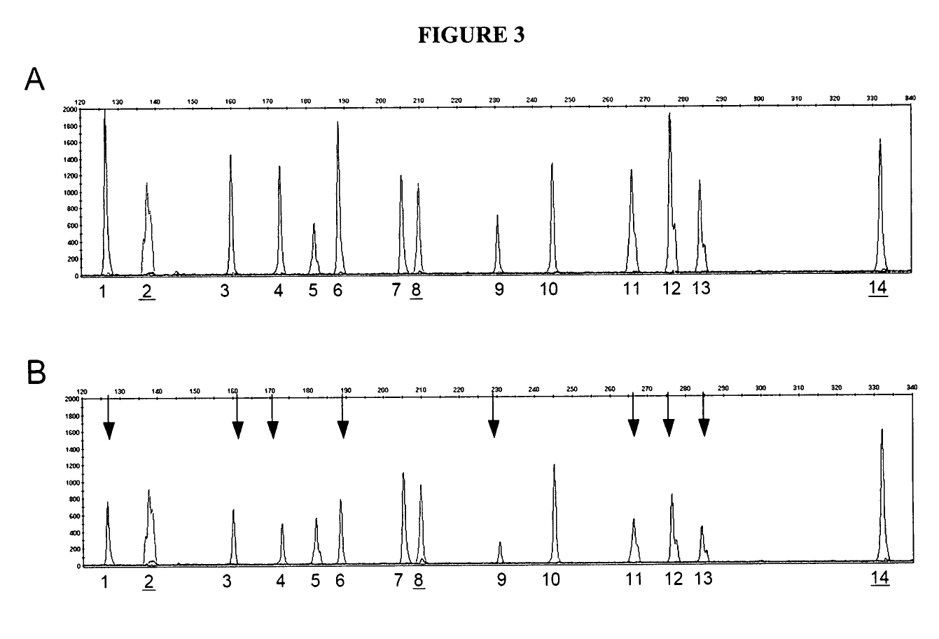

Exemplary electropherograms using: (A) a sample that is wild-type at the α-globin locus; and (B) a sample that is heterozygous for the -FIL deletion, is shown in FIG. 3. The “X” axis represents the number of bases, from 120 bases to 340 bases; the “Y” axis represents the level of fluorescence, from 0 to 2000 units.

The wild-type electropherogram in FIG. 3A shows 14 peaks, corresponding to the “peak number” noted in Column 1 of TABLE 9, above. FIG. 3B shows the same 14 peaks, however, the size of some of the peaks, which corresponds to the dye intensity, is reduced by about 50%. The black arrows in FIG. 3B show the peaks which are reduced in the -FIL mutant. The peaks are smaller (less fluorescence) because there is one less copy of each target in the -FIL mutant sample, and fewer amplicons are produced resulting in less fluorescence. The -FIL mutation results in a deletion of the HBA1, 2 and pseudo gene regions (see e.g., FIG. 1). Accordingly, peaks 1, 3, 4, 6, 11, 12, and 13, all HBA1 and HBA2 targets, are reduced in the mutant. Peak 9, which is an amplicon from the inter-pseudo gene region, is also reduced in the mutant. Control peaks 2, 8 and 14 are unaffected in the mutant as are peaks 5, 7 and 10, which are amplicons from the SRO and HS-40 region, regions not affected by the -FIL deletion (there are two copies of each SRO and HS-40 target in the mutant sample).\

### EXAMPLE 2

### Detection of Alpha Thalassemia in Patient Samples

Thirty-eight different DNA samples were tested using the compositions and methods described above in Example 1. The different samples were prepared using one of two different methods, Qiagen or Gentra, and using samples from two different sources, whole blood or prenatal samples from tissue-cultured cells obtained through the chorionic villi or amniocentesis. The samples had previously been typed using the gapped-DNA method or sequencing.

The samples and the preparation methods are presented in TABLE 10 below. “NS” indicates “no sample reagent control” and “ND” indicates a no DNA control.

As used in this experiment, “WT/WT” refers to the absence of rearrangements in the α-globin gene cluster. It does not exclude the presence of single nucleotide polymorphisms or a few base pair insertions and/or deletions.

TABLE 10

| | 1 | 2 | 3 | 4 | 5 |
|---|---|---|---|---|---|
| A | WT/WT | NS | alpha 3.7/WT | WT/WT | alpha 3.7/WT |
| B | MED/WT | ND | WT/WT | anti-3.7/WT | WT/WT |
| C | WT/WT | alpha 3.7/WT | alpha 3.7/alpha | WT/WT | alpha 3.7/WT |
| | | | 3.7 | | |
| D | WT/WT | WT/WT | SEA/WT | WT/WT | WT/WT |
| E | FIL/WT | alpha 3.7/WT | alpha 3.7/alpha | WT/WT | alpha 3.7/WT |
| | | | 3.7 | | |
| F | WT/WT | alpha 3.7/WT | SEA/WT | anti-3.7/WT | alpha 3.7/WT |
| G | SEA/WT | alpha 3.7/WT | SEA/WT | WT/WT | alpha |
| | | | | | 3.7/alpha 3.7 |
| H | WT/WT | alpha 3.7/WT | SEA/WT | WT/WT | alpha 3.7/WT |

| | | |
|---|---|---|
| Qiagen | Qiagen Prenatals | Gentra |

Each sample in the first set of DNA samples (i.e., columns 1 and 2 in TABLE 10) was extracted using automated Qiagen instruments and reagents. The WT/WT control for this set was sample A1 (bold, underlined). Each sample in the second set of samples (i.e., column 3 in TABLE 10) was extracted using an automated Qiagen method for prenatal samples. Sample B3 served as the WT/WT control for this set. Each sample in the third set of DNA samples (i.e., columns 4 and 5 in TABLE 10) was prepared with an automated Gentra extraction system, with sample A4 serving at the WT/WT positive control for this set.

Data analysis was performed using GeneMapper software (Applied Biosystems, Foster City, Calif.). Analysis was performed by comparing the electropherogram profiles of the WT/WT versus the test sample, as well as by comparing signal ratios to the internal controls. All samples were analyzed in duplicate.

All samples were genotyped correctly (electorpherograms not shown). The assay detected the presence of large deletions (e.g., the decrease in the level of fluorescence of amplicons within the deletion) but did not distinguish between the types of the deletions. For example, there were a total of seven large deletions: one MED/WT, one FIL/WT and five SEA/WT. By identifying a loss of one copy of HBA1 and one copy of HBA2, the assay accurately detected the presence of a large deletion and detected a dosage total of two in each of these samples (the two copies present on the wild-type allele). All of these deletions also encompass the pseudo genes. Accordingly, the amplicon between the pseudo genes also shows a decrease in fluorescence in these mutant samples.

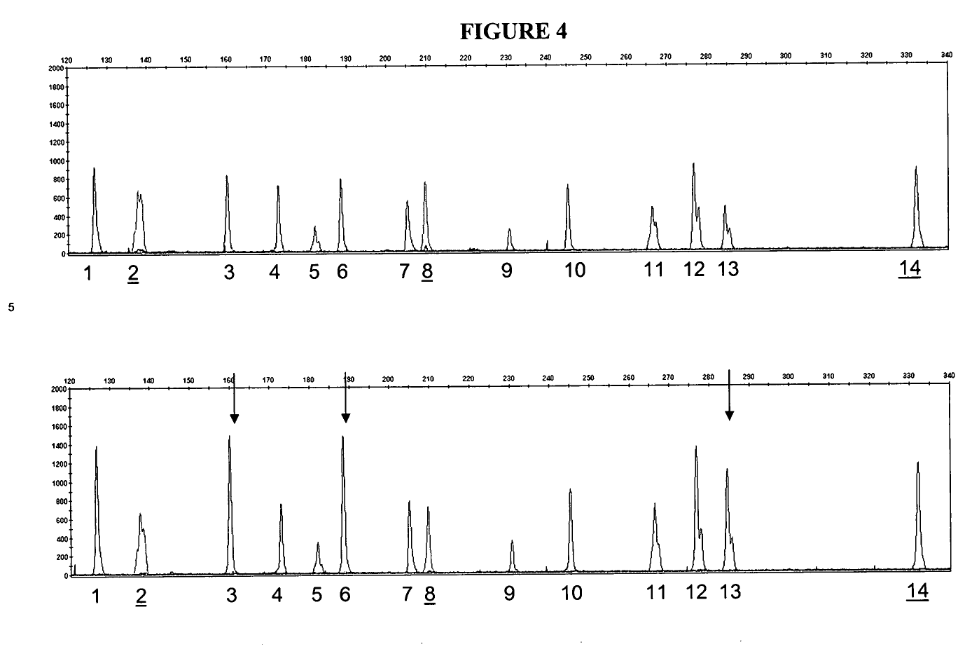

Additionally, the assay accurately detected the presence of two samples with five copies of α-globin-like gene regions (samples B4 and F4). In this particular sample, the extra copy is due to an anti −α3.7 rearrangement (i.e., an insertion/duplication of the α-3.7 region). In this case, an increase in the signals of the HBA1-5′, the HBA2-mid and HBA2-3′ over WT/WT levels was observed. FIG. 4 shows a wild-type and an anti −α3.7/WT electropherogram. Note the difference in the height of peaks 3, 6 and 13, relative to control peaks 2, 8 and 14 in the wild-type and mutant electropherograms. In the wild-type electropherogram, peaks 3 and 6 are roughly equal to the height of the control peaks 2, 8 and 14, while peak 13 is smaller than any of the controls. In the mutant electropherogram, peaks 3, 6 and 13 are roughly doubled in height relative to the controls. As shown in TABLE 9, peak 3 corresponds to an amplicon from the HBA1 5′ region; peak 6 corresponds to an amplicon from the HBA2 3′ region, and peak 13 corresponds to an amplicon from the HBA2-mid region. As shown in FIG. 1, this correlates with a duplication of the 3.7 kb region, which is generated as the −α3.7 deletion is formed on one chromosome due to recombination.

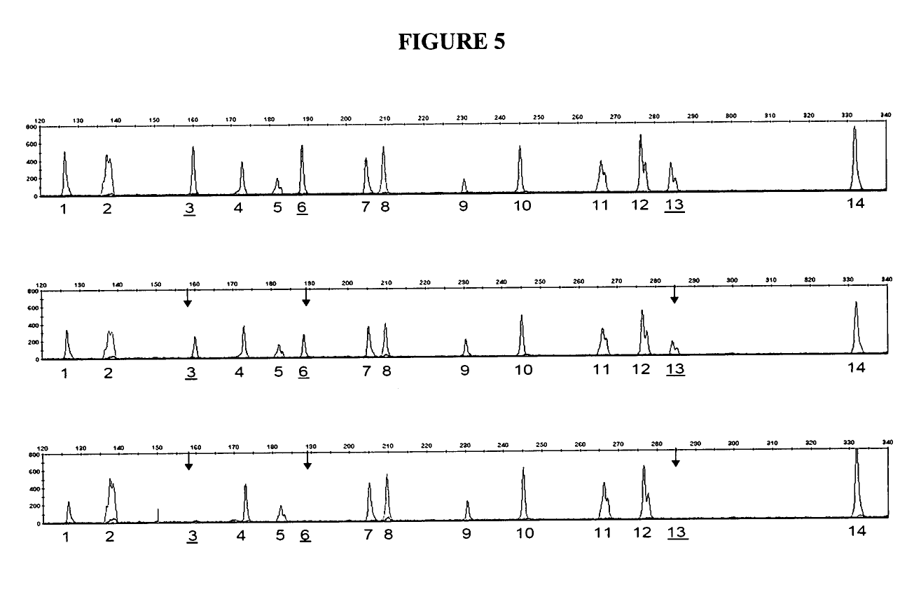

The assay also detected the presence of the homozygous −α3.7/−α3.7 mutants. FIG. 5 shows exemplary WT/WT, −α3.71WT, and −α3.7/−α3.7 electropherograms. As with the anti −α3.7 mutant discussed above, the relative height of peaks 3, 6, and 13 (underlined) are compared to the control peaks 2, 8, and 14. Note that in this WT/WT sample, peaks 3 and 6 are slightly higher than control peaks 2, 8 and just slightly lower than control peak 14, while peak 13 is slightly lower than all three controls. In the heterozygous −α3.7/WT sample, peaks 3 and 6 and 13 are lower than control peaks 2, 8 and 14, with peak 13 notably diminished as compared to the controls. In the homozygous −α3.7/−α3.7 mutant, peaks 3, 6 and 13 are absent.

The fragment “HBA1/2 common” (a fragment identical between HBA1 and HBA2) can be used as a guide for confirming large deletions or homozygous deletions that remove the 5′ end of HBA1 and/or HBA2, as the fluorescence intensity drops to about half the WT/WT signal. Note that for “HBA1/2 common,” the WT/WT genotype has four copies of this region, since it is present in both HBA1 and HBA2.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually.

That which is claimed is:

1. A method for determining α-globin gene dosage in a subject, said method comprising:
a. preparing a reaction mixture for amplifying nucleic acid comprising:
(i) genomic nucleic acids from a sample from the subject or a copy of genomic nucleic acids from a sample from the subject,
(ii) a first and a second set of primers capable of specifically amplifying a first and a second segment of HBA1,
(iii) a third and a fourth set of primers capable of specifically amplifying a first and a second segment of HBA2,
(iv) a fifth set of primers capable of specifically amplifying a first segment of SRO, and
(v) a sixth set of primers capable of amplifying a control sequence, wherein the control sequence is outside of the alpha-globin gene cluster;
wherein at least one primer from each set of primers comprises a label;
b. reacting the reaction mixture to generate amplification products;
c. measuring the amount of the various amplification products produced relative to the control amplified product, wherein the α-globin gene dosage is determined.

2. The method of claim 1, wherein said control sequence is selected from the group consisting of one or more of the following: a region of the hexosaminidase A gene; a region of the factor V gene; and a region of the factor II gene.

3. The method of claim 1, wherein the reaction mixture further comprises:
(vi) a seventh set of primers capable of specifically amplifying a third segment of HBA1;
(vii) an eighth set of primers capable of specifically amplifying a third segment of HBA2, and
(viii) a ninth set of primers capable of specifically amplifying a segment of HS-40;
wherein at least one primer of each of the seventh, eighth, and ninth primer sets comprises a label.

4. The method of claim 3, wherein the reaction mixture further comprises:
(ix) a tenth set of primers capable of specifically amplifying a sequence between the α-globin pseudo genes,
(x) an eleventh set of primers capable of specifically amplifying a second segment of SRO,
(xi) a twelfth set of primers capable of amplifying a second control sequence, wherein said second control sequence is outside of the alpha-globin gene cluster; and
(xii) a thirteenth set of primers capable of amplifying a third control sequence, wherein said third control sequence is outside of the alpha-globin gene cluster;
wherein at least one primer of each of the tenth, eleventh, twelfth, and thirteenth primer sets comprises a label.

5. The method of claim 4, wherein at least one primer set is selected from the group consisting of: SEQ ID NOs: 3 and 4; 18 and 20; 9 and 11; 5 and 6; 18 and 19; 9 and 10; 7 and 8; 16 and 17; 12 and 13; 14 and 15; 21 and 22; 23 and 24; and 25 and 26.

6. The method of claim 4, wherein at least one primer is selected from the group consisting of SEQ ID NOs:3-26.

7. The method of claim 1, wherein the detected amplification products are between 50 and 1000 nucleotides in length.

8. The method of claim 1, wherein the detected amplification products are between 100 and 500 nucleotides in length.

9. The method according to claim 1, further comprising a set of primers capable of amplifying a segment common to HBA1 and HBA2.

10. The method according to claim 9, wherein said set of primers comprise the oligonucleotides sequences set forth in SEQ ID NOs: 1 and 2.

11. A method for diagnosing a disease or confirming the diagnosis of a disease associated with or impacted by α-globin gene dosage in a subject comprising:
a. determining the number of functional α-globin gene copies present in the genomic nucleic acid of the subject; wherein determining comprises:
b. preparing a reaction mixture for amplifying nucleic acid comprising:
(i) genomic nucleic acids from a sample from the subject or a copy of genomic nucleic acids from a sample from the subject,
(ii) a first and a second set of primers capable of specifically amplifying a first and a second segment of HBA1,
(iii) a third and a fourth set of primers capable of specifically amplifying a first and a second segment of HBA2,
(iv) a fifth set of primers capable of specifically amplifying a first segment of SRO, and
(v) a sixth set of primers capable of specifically amplifying a control sequence, wherein the control sequence is outside of the alpha-globin gene cluster,
wherein at least one primer from each set comprises a label;
c. amplifying nucleic acids in the amplification mixture to generate amplification products;
d. measuring the amount of the various amplification products produced relative to the control amplified product to determine α-globin gene dosage, and
e. using the gene dosage to diagnose or confirm the diagnosis of a disease associated with or impacted by α-globin gene dosage.

12. The method of claim 11, wherein the disease is selected from the group consisting of α-thalassemia, hemoglobin H disease, Bart's hydrops fetalis, and β-thalassemia severity.

13. The method of claim 11, wherein said control sequence is selected from the group consisting of one or more of the following: a region of the hexosaminidase A gene; a region of the factor V gene; and a region of the factor II gene.

14. The method of claim 11, wherein the reaction mixture further comprises:
(vi) a seventh set of primers capable of specifically amplifying a third segment of HBA1;
(vii) an eighth set of primers capable of specifically amplifying a third segment of HBA2,
(viii) a ninth set of primers capable of specifically amplifying a segment of HS-40;
wherein at least one primer of each of the seventh, eighth, and ninth primer sets comprises a label.

15. The method of claim 14, wherein the reaction mixture further comprises:
(ix) a tenth set of primers capable of specifically amplifying a sequence between the α-globin pseudo genes,
(x) an eleventh set of primers capable of specifically amplifying a second segment of SRO,
(xi) a twelfth set of primers capable of amplifying a second control sequence, wherein said second control sequence is outside of the alpha-globin gene cluster; and
(xii) a thirteenth set of primers capable of amplifying a third control sequence, wherein said third control sequence is outside of the alpha-globin gene cluster;
wherein at least one primer of each of the tenth, eleventh, twelfth, and thirteenth primer sets comprises a label.

16. The method of claim 15, wherein at least one primer pair is selected from the group consisting of: SEQ ID NOs: 3 and 4; 18 and 20; 9 and 11; 5 and 6; 18 and 19; 9 and 10; 7 and 8; 16 and 17; 12 and 13; 14 and 15; 21 and 22; 23 and 24; 25 and 26.

17. The method of claim 15, wherein at least one primer is selected from the group consisting of SEQ ID NOs: 3-26.

18. The method of claim 11, wherein the detected amplification products are between 50 and 1000 nucleotides in length.

19. The method of claim 11, wherein the detected amplification products are between 100 and 500 nucleotides in length.

20. The method according to claim 11, further comprising a set of primers capable of amplifying a segment common to HBA1 and HBA2.

21. The method according to claim 20, wherein said set of primers comprise the oligonucleotides sequences set forth in SEQ ID NOs: 1 and 2.

22. A method for detecting if one or more mutations are present in the alpha-globin gene cluster of a subject, the method comprising:
a. preparing a reaction mixture for amplifying nucleic acid comprising:
(i) genomic nucleic acids from a sample from the subject or a copy of genomic nucleic acids from a sample from the subject,
(ii) a first and a second set of primers capable of specifically amplifying a first and a second segment of HBA1,
(iii) a third and a fourth set of primers capable of specifically amplifying a first and a second segment of HBA2,
(iv) a fifth set of primers capable of specifically amplifying a first segment of SRO,
wherein at least one primer from each set comprises a label;
b. amplifying nucleic acids in the amplification mixture to generate amplification products; and
c. detecting the amplification products to determine if one or more mutations are present in the alpha-globin gene cluster of the subject.

23. The method of claim 22, wherein the amplification reaction mixture further comprises:
(v) a sixth set of primers capable of specifically amplifying a control sequence, wherein the control sequence is outside of the alpha-globin gene cluster,
wherein at least one primer from each sixth set comprises a label.

24. The method of claim 23, wherein the control sequence is selected from the group consisting of one or more of the following: a region of the hexosaminidase A gene; a region of the factor V gene; and a region of the factor II gene.

25. The method of claim 23, wherein the amplification reaction mixture further comprises:
(vi) a seventh set of primers capable of specifically amplifying a third segment of HBA1;
(vii) an eighth set of primers capable of specifically amplifying a third segment of HBA2,
wherein at least one primer of each of the seventh and eighth primer sets comprises a label.

26. The method of claim 25, wherein the amplification reaction mixture further comprises:
(viii) a ninth set of primers capable of specifically amplifying a sequence between the α-globin pseudo genes,
wherein at least one primer from each ninth set comprises a label.

27. The method of claim 26, wherein at least one primer is selected from the group consisting of SEQ ID NOs: 3-26.

28. The method of claim 23, wherein said step of detecting includes evaluating the size and signal intensity of the amplified products relative to the control amplified products.

29. The method of claim 22, wherein the detected amplification products are between 100 and 500 nucleotides in length.

30. The method according to claim 23, further comprising a set of primers capable of amplifying a segment common to HBA1 and HBA2.

31. The method according to claim 30, wherein said set of primers comprise the oligonucleotides sequences set forth in SEQ ID NOs: 1 and 2.

32. The method according to claim 1 wherein:
said first segment of HBA1 amplified is from a 5′-end region of the HBA1 gene, wherein said 5′-end region corresponds to about nucleotides 165000 to about 166900 of GenBank Accession No. AE006462; and
said second segment of HBA1 amplified is from a middle region of the HBA1 gene, wherein said middle region corresponds to about nucleotides 166901 to 167270 or is from the 3′-end region of the HBA1 gene, wherein said 3′-end region corresponds to about nucleotides 167271 to about 167700.

33. The method according to claim 1 wherein:
said first segment of HBA2 amplified is from a 5′-end region of the HBA2 gene, wherein said 5′-end region corresponds to about nucleotides 161850 to about 163100 of GenBank Accession No. AE006462; and
said second segment of HBA2 amplified is from a middle region of the HBA2 gene, wherein said middle region corresponds to about nucleotides 163101 to 163470 or is from the 3′-end region of the HBA2 gene, wherein said 3′-end region corresponds to about nucleotides 163471 to about 163800.

34. An oligonucleotide selected from the group consisting of SEQ ID NOs: 1-26.

35. A kit comprising:
a. at least two oligonucleotide pairs capable of specifically hybridizing to and amplifying a first and second segment of the HBA1 gene;
b. at least two oligonucleotide pairs capable of specifically hybridizing to and amplifying a first and second segment of the HBA2 gene;
c. at least one oligonucleotide pair capable of specifically hybridizing to the SRO segment.

36. The kit of claim 35, further comprising:
d. at least one oligonucleotide pair capable of specifically hybridizing to and amplifying a segment of the HS-40 region;
e. at least one oligonucleotide pair capable of specifically hybridizing to and amplifying a segment of the region between the α-globin pseudo genes.

37. The kit of claim 36, further comprising:
f. at least one oligonucleotide pair capable of specifically amplifying a control sequence, wherein the control sequence is outside of the alpha-globin gene cluster.

38. The kit of claim 37, wherein at least one primer from each set of primers comprises a label.
